(19) United States
(12) Patent Application Publication
(10) Pub. No.: US 2020/0239830 A1
(43) Pub. Date: Jul. 30, 2020

(54) Methods of Mycological Biopolymer Production

(71) Applicant: Ecovative Design LLC, Green Island, NY (US)

(72) Inventors: Megan A. O'Brien, Halfmoon, NY (US); Alex Carlton, Troy, NY (US); Peter Mueller, Poestenkill, NY (US)

(73) Assignee: Ecovative Design LLC, Green Island, NY (US)

(21) Appl. No.: 16/773,272

(22) Filed: Jan. 27, 2020

Related U.S. Application Data
(60) Provisional application No. 62/796,640, filed on Jan. 25, 2019.

(51) Int. Cl. C12N 1/14; C12N 1/22; C12N 1/38

(52) U.S. Cl. CPC C12N 1/14; C12N 1/38; C12N 1/22

(57) ABSTRACT

Several types of non-agricultural lignocellulosic waste media are disclosed for the growth of mycological biopolymers. The growth medium is comprised of a substrate with a composition of appropriate Carbon, Nitrogen and mineral components including but not limited to lipids, proteins, and other inherent nutrition requisite for mycelial growth. Specific examples are (1) a lignocellulosic material, (2) a mineral based material, (3) a non-toxic, organic or inorganic, non-lignocellulosic material, (4) a synthetically sourced and produced material, (5) a whole tree (flourized), and (6) an agar media.

### CROSS-REFERENCE TO RELATED APPLICATIONS

This application claims the benefit of U.S. Provisional Patent Application No. 62/796,640, filed Jan. 25, 2019, the disclosure of which is considered part of, and is incorporated by reference in its entirety in, the disclosure of this application.

### TECHNICAL FIELD

This disclosure relates to methods for mycological biopolymer production, compositions prepared by said methods, and uses thereof.

### BACKGROUND

As is known, various techniques have been used to grow a mycological biopolymer material without the production of a fruiting body.

US Published Patent Application 2015/0033620, the entire content of which is hereby incorporated by reference in its entirety, describes techniques for growing a material comprising fungal mycelium, referred to as “mycological biopolymer.” As described therein, a mycological biopolymer product provided by the disclosed method is characterized as containing a homogenous polymer matrix that is comprised predominantly of fungal chitin and trace residues (e.g., beta-glucan, proteins). The mycological biopolymer is up-cycled from domestic agricultural lignocellulosic waste and is made by inoculating the domestic agricultural lignocellulosic waste substrate with a selected fungus in a container that is sealed off from the ambient environment external to the container. In addition to the substrate and fungal inoculum, the container contains a void space, which space is subsequently filled with a network of undifferentiated fungal mycelium. The biopolymer product grows into the void space of the container, filling the space with an undifferentiated mycelium comprising a chitin-polymer. The chitin-polymer-based mycelium is subsequently extracted from the substrate and dried. As further described in 2015/0033620, the environmental conditions for producing the mycological biopolymer product described therein, i.e. a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

As described in U.S. patent application Ser. No. 16/190,585, filed Nov. 14, 2018, another method of growing a biopolymer material employs incubation of a growth media comprised of a nutritive substrate and a fungus in containers that are placed in a closed incubation chamber with air flows passed over each container while the chamber is maintained with a predetermined environment characterized by parameters including relative humidity, temperature, carbon dioxide (CO₂) level and oxygen (O₂) level. The growth media in each container is incubated for a period of time sufficient for the fungus to digest the nutritive substrate and produce a mycelium biopolymer consisting entirely of fungal mycelium in each container.

As described in U.S. Ser. No. 16/519,384, filed Jul. 23, 2019, a panel of biopolymer material as described in U.S. Ser. No. 16/190,585, may be modified to generate a material with a custom texture, flavor, and nutritional profile for use as a foodstuff or a tissue scaffold. The method involves tailoring the density, morphology, and composition of the undifferentiated fungal material during growth and/or the use of post-processes, to improve mouth-feel and/or affinity toward flavors, fats, cellular cultures, or the like.

It is an object of the disclosure to provide alternative growth media for mycological biopolymer growth. It is another object of the disclosure to simplify the growing of mycological biopolymers. It is yet another object of the disclosure to provide mycological biopolymers with improved properties.

The present disclosure provides a growth medium other than domestic agricultural lignocellulosic waste, particularly, alternatives to corn stover, that will reliably elicit mycological biopolymer growth without inducing the formation of visible fungal fruiting bodies, such as a stipe, pileus, gill or pore structure.

In some aspects, this method involves the replacement of domestic agricultural lignocellulosic waste as the substrate base with a growth medium that capably supports growth, but that is supplied more readily, consistently and efficiently, that is produced more cleanly and with less labor associated with its production, that can provide alternate sources of nutrition or of other types of growth support, that can be tailored to suit growth of particular species of fungus, or that can be produced repeatedly as a synthetically sourced material. Our evaluation of replacements for domestic agricultural lignocellulosic waste led to the discovery of methods of tailoring the mycelial growth to generate mycological biopolymers having particular growth morphologies and improved properties, including bulbing, density differentiation and/or gradation through mat height.

In a general aspect, the disclosure provides, a method of growing a mycelial mat comprising the steps of, providing a growth medium of non-agricultural lignocellulosic waste media; adding a nutrient for fungal growth and a fungal inoculum to said growth medium thereafter placing said growth medium in a closed incubation chamber; maintaining said closed incubation chamber with a predetermined environment of humidity, temperature, carbon dioxide and oxygen sufficient to prevent the full differentiation of the fungus into a mushroom without producing a stipe, cap, or spores; and incubating the growth media in each said container for a period of time sufficient for said fungus to digest said nutritive substrate and produce a mycelium mat consisting entirely of fungal mycelium on said growth medium.

The growth media may be a lignocellulosic material having a particle size. The lignocellulosic material may have a particle size of no more than ¼ inch. The lignocellulosic material may have a particle size in a range of from ¼ inch to 2 inches.

The growth media may be a mineral based material. The mineral based material may be at least one of vermiculite, perlite, soils and chalk.

The growth media may be a non-toxic non-cellulosic material. The non-toxic non-cellulosic material may be one of at least one of hydroponic media, gels, polyacrylates, plastics and agars. The non-toxic non-cellulosic material may be agar having an additive therein to support the growth of mycelium. The fungal inoculum may be spread across the agar growth media.

The growth media may be a synthetically sourced and produced material. The material may be a plastic. The material may be polyurethane.

The method may comprise placing the nutrient, fungus and growth medium in at least one growth form prior to said step of placing said growth medium in a closed incubation chamber.

The method may comprise placing the nutrient, fungus and growth medium on at least one growth support surface prior to said step of placing said growth medium in a closed incubation chamber.

The method may further comprise the step of doping said substrate with at least one of a nitrite, supplement and a drug. The supplement may be a food preservative. The drug may be allicin derived from garlic.

The method may further comprise the step of adding a compound to the growth medium to inhibit contaminants. The compound may be at least one of tannins to retard ascomycetes, cinnamaldehyde to inhibit bacteria, and sorbate to inhibit yeast.

In a general aspect, the disclosure provides a mycelium mat made in accordance with the foregoing method(s).

In a general aspect, the disclosure provides, a method of preparing a mycological biopolymer comprising, providing a mixture, the mixture comprising a substrate and a fungal inoculum. The fungal inoculum comprises a fungus. The method further comprises incubating the mixture as a solid-state culture in a growth environment of moisture, temperature, carbon dioxide (CO₂) and oxygen (O₂) sufficient to support fungal growth comprising fungal mycelial growth; thereby providing a product comprising the mycological biopolymer. The growth environment may inhibit the formation of a fruiting body. The fungal growth may consist essentially of the fungal mycelial growth. The fungal growth may consist of the fungal mycelial growth. The incubation occurs over a period of time sufficient for the fungus to produce the product comprising the mycological biopolymer. The mycological biopolymer product so obtained does not contain a visible fruiting body, such as a stipe, pileus, gill or pore structure. The product is an aerial mycological biopolymer. The mycological biopolymer is not appressed. The aerial mycological biopolymer has a thickness. The thickness may be greater than about 0.125 inch, or at least about 0.25 inch. The thickness may be at least about 0.5 inch, at least about 0.75 inch or at least about 1 inch. The thickness may be at most about 4, 6, 8 or 10 inches.

The product comprising the mycological biopolymer may further comprise a portion of the substrate, any optional nutritional supplement, or a combination thereof.

The method may further comprise removing the mycological biopolymer from at least a portion of the substrate, or from any remaining substrate.

The product may consist essentially of the mycological biopolymer.

The product may consist of the mycological biopolymer.

The fungus may be of a type which produces edible mushrooms. Thus, the fungus may be one of Agaricus sp., Lentinula sp., Flammulina sp., Laetiporus sp., Pleurotus sp., Hericium sp., Morchella sp.,Agrocybe sp. or Hypsizygous sp. The fungus may be Pleurotus ostreatus.

The fungus may be a white rot fungus. Thus, the fungus may be one of Phanerochaete sp., Ceriporiopsis sp., Ganoderma sp., Omphalotus sp., Trametes sp., Polyporellus sp. or Schizophyllum sp. The fungus may be Ganoderma tsugae. The fungus may be Ganoderma G. resinaceum.

The substrate may be a natural substrate. The natural substrate may comprise a lignocellulosic material. The lignocellulosic material may comprise a plant or wood material. The plant or wood material may be purposefully harvested for use in the production of the mycological biopolymer. The lignocellulosic material may comprise hemp, maple, corn, kenaf, canola, soy straw, wheat straw, seed or seed husk material; or any combination thereof. The seed may be sunflower seed, walnut or poppy seed; or any combination thereof. The lignocellulosic material may be a wood material comprising a hardwood or softwood material. The hardwood or softwood may be of the genus Acer, Quercus, Populus, Abies or Pinus. The lignocellulosic material may comprise a wood flour, such as maple wood flour, or a plant flour, such as soy flour. The lignocellulosic material is not an agricultural waste product. The lignocellulosic material is not corn stover.

The natural substrate may comprise a cellulosic material. The cellulosic material may be a lignose-free material. The cellulosic material may contain plant fiber. The plant fiber may be obtained from cotton (Gossypium sp.), hemp (Cannabis sp.), flax (Linum sp.) or jute (Corchorus sp.). The cellulosic material may be pet bedding, paper, cardboard, card stock, cotton, linen or textile; or a combination thereof.

The natural substrate may comprise an inorganic material such as a mineral or mineral-based material. The mineral or mineral-based material may be vermiculite, perlite, soil, chalk, gypsum, clay, sand, rockwool or growstones or a combination thereof. The mineral or mineral-based material may be a lignose-free material.

The substrate may comprise a synthetic material. The synthetic material may be a plastic. The synthetic material may be a synthetic organic polymer such as a polyethylene, a polypropylene, a polyvinyl chloride, a polystyrene, a polyacrylate, a nylon, a polytetrafluoroethylene (e.g.,Teflon™), a polyamide, a polyester, a polysulfide, a polycarbonate, a polythene or a polyurethane. The synthetic organic polymer may contain one or more heteroatoms. The synthetic organic polymer may be a polyurethane, or more particularly, a thermoplastic polyurethane. The synthetic material may be obtained from a recycled material.

The substrate may comprise an artificial material. The artificial material may be an alginate, such a sodium alginate, rayon or agar-agar.

In some aspects, the substrate may be provided as particles characterized as having a particle size.

The particle size may be at most about 0.25 inch in diameter, or less than 0.25 inch in diameter.

The particle size is at most about 0.125 inch in diameter, or less than about 0.125 inch in diameter.

The particle size may be at most about 0.01 inches in diameter, or at most about 0.007 inch in diameter.

The particle size may be at least about 0.25 inch, or greater than 0.25 inch in diameter, and at most about 2 inches in diameter.

The method may further comprise sizing the substrate to the desired particle size prior to providing the mixture.

In some aspects, the substrate is a monolithic substrate.

The monolithic substrate may be a contiguous porous solid, such as a log, a slab of wood, textile or a solidified porous gel medium; or a combination thereof. The monolithic substrate may be a continuous woven textile or a continuous non-woven textile, which may comprise rockwool, cotton, wood fiber or polyester fiber; optionally, the continuous textile is provided in the form of a mat. The monolithic substrate may comprise a combination of two or more monolithic substrates.

The substrate is a solid or a gel. The substrate may be a liquid, provided that the culture is not a submerged culture.

Any substrate contained in the mixture may be a non-toxic substrate.

Any substrate contained in the mixture may be a sterile substrate. Thus, in some embodiments, the method further comprises sterilizing the substrate prior to providing the mixture. The sterilization may be heat sterilization, steam sterilization, or irradiation with electromagnetic radiation; optionally, the electromagnetic radiation comprises gamma rays, X-rays, UV or UV-visible radiation.

The mixture may consist essentially of the substrate and the fungal inoculum.

The mixture may consist of the substrate and the fungal inoculum.

The method may further comprise inoculating the substrate with the fungal inoculum to provide the mixture.

The mixture may further contain one or more nutritional supplements, in addition to the substrate and the fungal inoculum. The nutritional supplement may be a lignocellulosic material. The lignocellulosic material may comprise seed, seed husks or both.

The method may further comprise preparing a blend containing the substrate and the nutritional supplement, and then inoculating the blend with the fungal inoculum to provide the mixture.

The fungal inoculum may be a seed-supported fungal inoculum. The fungal inoculum may be a seed-saw dust mixture fungal inoculum, or another commercially available fungal inoculum.

The method may comprise terminating the incubation prior to the fungus forming a visible fruiting body. The termination of the incubation may comprise exposing the incubation mixture comprising the solid-state culture to a terminal environment, wherein the terminal environment is different from the growth environment. The terminal environment may have one or more conditions that differ from the corresponding growth environment conditions, such as the moisture content, temperature, carbon dioxide content and/or oxygen content. Exposing the incubation mixture comprising the solid-state culture to the terminal environment may comprise physically moving the mixture comprising said solid-state culture from the growth environment to the terminal environment. Exposing the incubation mixture comprising the solid-state culture to the terminal environment may alternatively comprise modifying one or more conditions of the growth environment, thereby providing the terminal environment. Terminating the incubation may comprise restoring the growth environment to ambient environmental conditions.

The mixture may be incubated in a tool. The tool may be essentially as described in US2015/2015/0033620, the entire content of which is hereby incorporated by reference in its entirety. The method may further comprise placing the mixture inside a tool. The tool may include a container. The mixture may be placed inside the container. The container may maintain a void volume after placing the mixture into the container. The tool may further comprise a lid having an opening.

The mixture may be incubated on a planar surface, such as a tray, a sheet, a table or a conveyer belt. The method may further comprise placing the mixture on the planar surface. The planar surface may be contained in a room, wherein the room may provide the growth environment.

During the incubation, the carbon dioxide may be present at a level ranging from about 3% (v/v) to about 7% (v/v), or from about 5% (v/v) to about 7% (v/v). The growth environment temperature may range from about 55° F. to about 100° F., or from about 85° F. to about 95° F. The period of time may be up to about 3 weeks, or up to about 2 weeks.

In a general aspect, the disclosure provides, a product prepared by a method of preparing a mycological biopolymer, as disclosed herein.

The product may be an edible product. The product may be suitable for use as a foodstuff, or for use in the manufacture of a foodstuff.

The product may be suitable for use as a tissue scaffold, or for use in the manufacture of a tissue scaffold.

The product may be used in the manufacture of a finished product.

### BRIEF DESCRIPTION OF THE DRAWINGS

FIG. 1 illustrates a photograph of a top view of a mycelial mat growth in accordance with the present disclosure, where the tissue is largely homogenous in texture.

FIG. 2 illustrates a photograph of a top view of another mycelial mat growth in accordance with the present disclosure, where the tissue is largely heterogeneous in texture.

FIG. 3 illustrates a graph of mass of a mycelial mat growth versus the substrate and chosen fungal inoculum.

### DETAILED DESCRIPTION

The present disclosure is directed to methods for mycological biopolymer production. Also disclosed are products prepared by said methods and uses thereof.

The following disclosure illustrates aspects of the methods, products and uses embodied in the claims.

In some aspects, there is provided a method of growing a mycological biopolymer. The term mycological biopolymer as disclosed herein is interchangeable with “mycelial mat.” In some embodiments, the method comprises providing a substrate to support growth of the mycological biopolymer. In some embodiments, the substrate is a natural substrate, a synthetic substrate or an artificial substrate, as disclosed herein. As used herein, “substrate”, refers to a media that supports growth of a mycological biopolymer, and expressly excludes an agricultural lignocellulosic waste material such as corn stover.

In some embodiments, the natural substrate for supporting growth of the mycological biopolymer comprises a lignocellulosic material. As used herein, “lignocellulosic material” refers to biomass comprising in part cellulose and lignin. In some embodiments, the lignocellulosic material is, or is derived from, a plant or wood material. In some embodiments, the plant or wood material is purposefully harvested for use in the production of the mycological biopolymer. As noted, the lignocellulosic substrate is not an agricultural waste product, such as corn stover. Nonlimiting examples of suitable lignocellulosic substrate comprising or obtained from plant or wood material. Nonlimiting examples of plant material include hemp, maple, corn, kenaf, canola, soy straw, wheat straw, seed, seed husk, and the like. In some embodiments, seed(s), seed husks, or both include sunflower seed, walnut, poppy seed, etc. Nonlimiting examples of tree material include hardwoods and softwoods, including from the genus Acer, Quercus, Populus, Abies, Pinus, and the like. In some embodiments, the lignocellulosic material comprises a wood or plant flour. In a nonlimiting embodiment, the wood flour is maple wood flour. In some embodiments, the plant flour is soy flour. In some embodiments, the method of growing a mycological biopolymer comprises providing a substrate comprising, consisting of, or consisting essentially of, the lignocellulosic material.

In some embodiments, the natural substrate for supporting growth of the mycological biopolymer is a cellulosic material. As used herein, “cellulosic material” refers to biomass comprising primarily of cellulose. In some embodiments, the cellulosic material is a lignose-free material. Nonlimiting examples of a cellulosic material include plant fibers such as derived from cotton (Gossypium sp.), hemp (Cannabis sp.), flax (Linum Sp.), and jute (Corchorus Sp.). Other nonlimiting examples include pet bedding, paper, cardboard, card stock, cotton, linen and/or textile. In some embodiments, the method of growing a mycological biopolymer comprises providing a substrate comprising, consisting of, or consisting essentially of, the cellulosic material.

In some embodiments, the natural substrate for supporting growth of the mycological biopolymer is an inorganic material. In some further embodiments, the inorganic material is a mineral or mineral-based material. Nonlimiting examples of a mineral or mineral-based material include vermiculite, perlite, soils, chalk, gypsum, clay (optionally in the form of beads), sand, rockwool, expanded clay, growstones, or the like. In some embodiments, the mineral or mineral-based substrate is a lignose-free material. In some embodiments, the method of growing a mycological biopolymer comprises providing a substrate comprising, consisting of, or consisting essentially of, the inorganic material, such as the mineral or mineral-based material.

In other embodiments, the substrate supporting growth of the mycological biopolymer is a synthetic material. In some embodiments, the synthetic material is a plastic. In some embodiments, the synthetic material is a synthetic polymer. In some embodiments, the synthetic polymer is a synthetic organic polymer. In some embodiments, the synthetic organic polymer is a polyethylene, a polypropylene, a polyvinyl chloride, a polystyrene, a polyacrylate, a nylon, a polytetrafluoroethylene (e.g.,Teflon™), a polyamide, a polyester, a polysulfide, a polycarbonate, a polythene or a polyurethane. In some embodiments, the synthetic organic polymer contains one or more heteroatoms (e.g., nitrogen and/or sulfur), including but not limited to a polyamide, a polyester, a polyurethane, a polysulfide or a polycarbonate. In some further embodiments, the synthetic organic polymer is a polyethylene, or more particularly, a high-density polyethylene or a low-density polyethylene. In some other embodiments, the synthetic organic polymer is a polyurethane, or more particularly, a thermoplastic polyurethane. In some embodiments, the synthetic organic polymer is a nylon, or more particularly nylon 6 or nylon 6,6. In some embodiments, the synthetic material is obtained from recycled materials. In some embodiments, the method of growing a mycological biopolymer comprises providing a substrate comprising, consisting of, or consisting essentially of, the synthetic material.

In yet other embodiments, the substrate supporting growth of the mycological biopolymer is an artificial material. Nonlimiting examples of an artificial material include an alginate (e.g., an alginate salt such as sodium alginate), rayon (e.g., rayon fiber, such as Viscose), agar or agar-agar, and the like. In some embodiments, the method of growing a mycological biopolymer comprises providing a substrate comprising, consisting of, or consisting essentially of, the artificial material.

In some embodiments, the substrate is non-toxic. As used herein, a non-toxic substrate refers to a substrate that does not inhibit the growth of, or which does not cause the death of the fungus. Thus, the substrate does not contain a toxic substance at a sufficiently high concentration that would inhibit the growth or cause the death of the fungus. In some embodiments, a non-toxic substrate is a substrate that is free of heavy metals, detergents and/or other cytotoxic agents.

In some aspects of the disclosure, the substrate for supporting the growth of the mycological biopolymer is characterized according to a particle size. Various methods of preparing a substrate of a desired particle size are known in the art. Typically, prior to providing the substrate supporting the growth of a mycological biopolymer of the present disclosure, a bulk substrate is provided, which may be sized by methods known in the art. Nonlimiting examples of sizing the substrate include passing a bulk substrate material through a mesh, screen, sieve or the like, use of a shaker table or vibrating screen sorter, etc., grinding, milling, etc., to obtain a substrate of the desired particle size. In some embodiments, the bulk substrate is treated and sized according to methods known in the art to provide the substrate in the form of a wood flour or flour-like material. In some embodiments, the sizing of the substrate to the desired particle size is performed prior to a further processing step, such as a sterilization or pasteurization.

In some embodiments, the particle size is at most about 0.01 inches in diameter. In some embodiments, the particle size is less than about 0.01 inch in diameter; optionally, about 0.007 inch in diameter or less. In some embodiments, the particles are wood or plant flour particles.

In some embodiments, the particle size is at most about 0.125 inch in diameter. In some embodiments, the particle size is less than about 0.125 inch in diameter.

In some embodiments, the particle size is at most about 0.25 inch in diameter. In some embodiments, the particle size is less than about 0.25 inch in diameter.

In some embodiments, the particle size larger, for example, the particle size is at least about 0.25 inch, or greater than 0.25 inch in diameter. In some further embodiments, the particle size ranges from about 0.25 inches to about 2 inches in diameter.

In some embodiments, the particle size refers to a maximum particle diameter. In some embodiments, the particle size refers to a mean particle diameter. In other embodiments, the particle size refers to a median particle diameter.

In some aspects, the substrate for supporting the growth of the mycological biopolymer is characterized according to its form or configuration. In some embodiments, the substrate is a solid. In some embodiments, the substrate is a gel. In some embodiments, the substrate is a liquid, provided that upon or after inoculation with a fungal inoculum, the resulting culture is not a submerged culture. In some embodiments, the substrate is provided as particles, as described herein. In other embodiments, the substrate is provided as a monolithic substrate, such as a contiguous porous solid such as a log, a slab of wood, a solidified porous gel media, or the like. In some more particular embodiments, two or more monolithic substrates are combined (e.g., a log slab penetrating into or through a solidified porous gel media). In other embodiments the substrate is provided as a continuous woven or non-woven textile, such as a rockwool mat, a nonwoven cotton mat, a wood fiber mat, a polyester fiber mat, or the like.

In some embodiments, the substrate is sterilized. In some embodiments, the sterilization is performed prior to inoculation of the substrate with fungal inoculum. Nonlimiting examples of substrate sterilization methods include heat sterilization, steam sterilization (such as autoclaving), or exposure to electromagnetic radiation (such as but not limited to UV, gamma or X-rays).

As disclosed herein, substrate identity, particle size or density may be selected and/or tuned to elicit particular growth morphologies or textures, such as bulbing, density differentiation, or gradation through mat height. As used herein, “bulbing” refers to a localized densification of the aerial tissue that results in a spheroidal tissue mass.

In some aspects, the growth environment is such that the carbon dioxide is present at a level ranging from about 3% (v/v) to about 7% (v/v). In some other embodiments, the carbon dioxide level ranges from about 5% (v/v) to about 7% (v/v).

In some aspects, the growth environment temperature ranges from about 55° F. to about 100° F. In some embodiments, the growth environment temperature ranges from about 85° F. to about 95° F.

In some aspects, the growth environment inhibits the formation of a fruiting body.

In some aspects, the incubation time is up to about 3 weeks. In some other aspects, the incubation time is up to about 2 weeks.

In some embodiments, the substrate comprises one or more nutritional agents, without requiring the addition of a further nutritional supplement. Thus, in some aspects, the one or more nutritional agents is inherently present in the substrate obtained or sourced for its intended use in supporting the growth of the mycological biopolymer. In some embodiments, the one or more nutritional agents is one or more organic components (e.g., components or compounds containing carbon oxygen and optionally nitrogen), including but not limited to one or more lipids, simple and/or complex carbohydrates and/or proteins. In some embodiments, the one or more nutritional agents present in the substrate includes one or more mineral(s), vitamin(s), coenzyme(s), element(s), and the like.

In some aspects, the method of growing a mycological biopolymer of the present disclosure comprises providing one or more nutritional supplements to supplement any nutritional value that may be present in the substrate. In some embodiments, the nutritional supplement includes lignocellulosic materials having high fat content, such as seeds. Thus, in some embodiments, the nutritional supplement comprises seed(s), seed husks or both (e.g., sunflower, walnut, poppy seed, etc.). As noted above, in some embodiments, the seeds and/or seed husks are provided as components of the substrate.

In some aspects, the method of growing a mycological biopolymer of the present disclosure comprises providing a fungal inoculum comprising a fungus. In some embodiments, the fungus is of a type which produces edible mushrooms. Nonlimiting examples of a type of fungus which produces edible mushrooms include Agaricus sp., Lentinula sp., Flammulina sp., Laetiporus sp., Pleurotus sp., Hericium sp., Morchella sp., Agrocybe sp. and Hypsizygous sp. In some emodiments, the fungus is Pleurotus ostreatus. In some other embodments, the fungus is a white rot fungus. Nonlimiting examples of a white rot fungus include Phanerochaete sp., Ceriporiopsis sp., Ganoderma sp., Omphalotus sp., Trametes sp., Polyporellus sp. and Schizophyllum sp. In some embodiments, the fungus is Ganoderma tsugae. In some other embodiments, the fungus is Ganoderma G. resinaceum. ln some aspects, the method of growing a mycological biopolymer of the present disclosure optionally comprises providing one or more additives. In some embodiments, the substrate is doped with a specific compound, including but not limited to a nitrite, a nutritional supplement, a dietary supplement, a preservative, a mineral or a drug. The specific compound may then be bio-accumulated by the fungal mycelium tissue, and may further become a functional component of the finished product containing the mycological biopolymer. In a nonlimiting example, a finished product may be a food, and the additive may be a food preservative. In another example, the finished product may be a wound dressing, and the additive may be a drug, for example, an antimicrobial agent. The wound dressing may thus be impregnated with the drug. In a further nonlimiting example, the additive is an antimicrobial agent, such as a tannin, cinnamaldehyde or sorbate.

In some embodiments, the additive is a nutritional agent or supplement, which may be added to the substrate to support mycelial growth during the production of the mycological biopolymer. In some aspects, the substrate (e.g., an agar media) may be formulated through the addition of nutrition, including but not limited to supplements or minerals. Such additives may support mycelial growth. An alternative or additional additive may be a drug. In one embodiment, the drug is forskolin In another embodiment, the drug is 10-oxo-trans-8-decenoic acid.

The present disclosure provides for methods of preparing mycological biopolymer-based products and essentially pure mycological biopolymers. In some aspects, the method comprises providing a mixture, the mixture containing a substrate as disclosed herein, and further containing a fungal inoculum, the fungal inoculum containing a fungus. Suitable fungi for preparing the mixture are disclosed herein. In some further embodiments, the method comprises incubating the mixture as a solid-state culture for a period of time in a growth environment. In some embodiments, the growth environment is one having moisture, a temperature, carbon dioxide (CO₂) and an oxygen (O₂). The growth environment of moisture, temperature, carbon dioxide (CO₂) and oxygen (O₂) is sufficient to support fungal growth comprising mycelial growth. In some aspects, the fungal growth consists essentially of mycelial growth. In some further aspects, the fungal growth consists of fungal mycelial growth. The growth environment may inhibit the formation of a fruiting body. The fungal growth occurring during the incubation in the growth environment excludes the formation of visible fruiting bodies.

Optionally, the mixture further comprises a nutritional agent. In such embodiments, the method of preparing the mycological biopolymer of the disclosure further comprises preparing a blend, the blend containing the substrate, as disclosed herein, and the nutritional agent. In a nonlimiting example of a method of the disclosure, a fungal inoculum is added to the blend to provide the mixture. Further agents or additives such as those disclosed herein may be added to the mixture.

In some further embodiments, the method further comprises sterilizing the substrate or blend prior to providing the mixture. In some embodiments, method comprises sterilizing the substrate or blend using heat sterilization, steam sterilization, or irradiation with electromagnetic radiation, such as gamma rays, X-rays, UV or UV-visible radiation.

In some further embodiments, the method further comprises placing the mixture in a tool. A suitable tool includes a tool is essentially as described in US2015/2015/0033620, the entire content of which is hereby incorporated by reference in its entirety. In some embodiments, a suitable tool is an incubation chamber. In other embodiments, the method comprises placing the mixture on a planar surface. Nonlimiting examples of a planar surface for placing the mixture include a tray, a sheet, a table, a conveyer belt and the like. In another aspect, the method comprises exposing the mixture contained in the tool or placed on the planar surface to growth environmental conditions, thereby initiating an incubation period. In yet further aspects, the method further comprises incubating the mixture as a solid-state culture for a period of time in the growth environment.

In some aspects, the method excludes submerging the culture in a liquid.

In yet further aspects, the method comprises terminating the incubation prior to the fungus forming a visible fungal fruiting body. Thus, in some embodiments, the method comprises terminating the incubation prior to the formation of a visible stipe, pileus, gill or pore structure associated with the mycological biopolymer. In some embodiments, terminating the incubation comprises modifying one or more of the growth environmental conditions.

In still other aspects, the method of the disclosure provides a product comprising a mycological biopolymer. In some embodiments, such as when the product further comprises residual substrate, the method optionally further comprises removing the substrate or a decomposition product thereof from the mycological biopolymer.

The present disclosure further provides for compositions of matter. In some aspects of the disclosure, there is provided a product obtained from a method of preparing a mycological biopolymer, as disclosed herein. The methods of the present disclosure provide products characterized as aerial mycological biopolymers. The aerial mycological biopolymers may be characterized as not appressed. In some embodiments, and according to methods disclosed herein, the mycological biopolymer product does not contain a visible fruiting body, such as a stipe, pileus, gill or pore structure. In some embodiments, the mycological biopolymer is characterized by its thickness. In some embodiments, the mycological biopolymer obtained by a method of the disclosure has a thickness of greater than about 0.125 inch. In some further embodiments, the mycological biopolymer has a thickness of at least about 0.25 inch. In yet some further embodiments, the mycological biopolymer has a thickness of at least about 0.5 inch, at least about 0.75 inch or at least about 1 inch. Any of the foregoing mycological biopolymers may have a thickness of at most about 4, about 5, about 6, about 7, about 8, about 9 or about 10 inches.

In some embodiments, the product comprises the mycological biopolymer. In some embodiments, the product further comprises an additive, such as an additive as disclosed herein. In some other embodiments, the product comprises a portion of the substrate, a portion of any nutritional additive or supplement that was added to the mixture, or a combination thereof.

In some other embodiments, the product consists essentially of the mycological biopolymer. In some further embodiments, the product consists of the mycological biopolymer.

The present disclosure further provides for uses of the products of the present disclosure. In some embodiments, a product prepared by a method of the present disclosure is suitable for use as a foodstuff. In some other embodiments, a product prepared by a method of the present disclosure is suitable for use as a tissue scaffold.

In some embodiments, the present disclosure provides for the use of a product of the present disclosure in the manufacture of a foodstuff. In other embodiments, there is provided the use of a product of the present disclosure in the manufacture of a tissue scaffold. In yet other embodiments, there is provided the use of a product of the present disclose in the manufacture of a finished product.

### EXAMPLES

The following sets forth several examples of using a substrate, or a blend comprising a substrate and optional nutritional agents, for growing a mycological biopolymer of the present disclosure.

### Example 1

1. A lignocellulosic material that is finely ground to less than ¼ particle size, including hemp, maple, corn, kenaf, canola, soy straw, wheat straw, seed husk or similar plant or wood material purposefully harvested for use in mycological biopolymer production, is sterilized.

2. Nutrients for fungal growth and fungal inoculum are added and the mixture is placed into a growth form designed to incite growth of mycological biopolymer material where seed-supported fungal inoculum addition can range from 300 g to 1900 g per standard tray volume of 280 cubic inches; and where nutrition type can vary so long as the nutrition type provides appropriate protein, fat and/or sugar additions within the range of 20 and 40% (protein), 10 and 40% (fat), and 6 to 20% (complex or simple carbohydrate (sugar)) respectively.

3. The filled growth form is placed into a closed environmental chamber as described in prior disclosures (US Published Patent Application 2015/0033620; and US application U.S. Ser. No. 16/190,585) and grown until the desired mycological biopolymer growth is achieved.

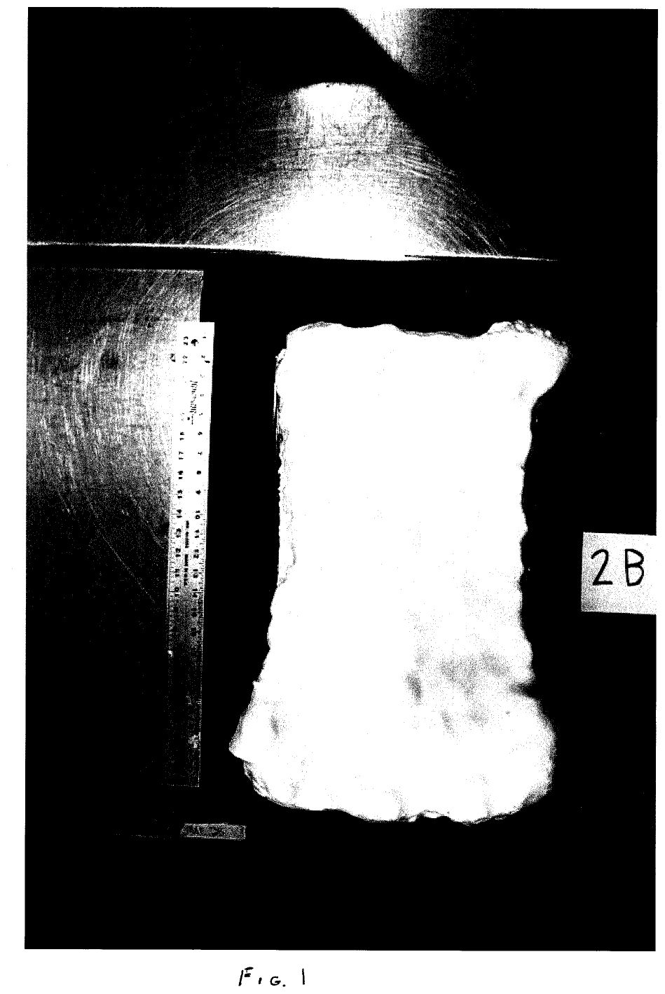

Referring to FIG. 1, the aerial mycelium growth produced on a ligno-cellulosic wood flour according to Example 1 is characterized in having generally homogeneously produced tissue texture. As such, the aerial mycelial growth may function as a scaffold as described in U.S. Ser. No. 16/519,384.

### Example 2

1. A mineral based material that is of consistent particle size less than ¼, including at least one of vermiculite, perlite, soils, chalk or the like, is cleaned through sterilization methods.

2. Nutrients and fungal inoculum are added and the mixture is placed into a growth form designed to incite growth of mycological biopolymer material

3. The filled growth form is placed into an environmental chamber as described in Example 1 and grown until the desired mycological biopolymer growth is achieved

### Example 3

1. A non-toxic, organic or inorganic non-cellulosic material that is of consistent size and shape or is of consistent texture or being, such as at least one of hydroponic media, gels, polyacrylates, plastics, and agars, is utilized in its existing state, or is cleaned through sterilization.

2. Nutrients are either added to the base material or are extant within the material, and fungal inoculum is added.

3. The mixture is placed into a growth form designed to incite growth of mycological biopolymer material.

4. The filled growth form is placed into an environmental chamber as described in Example 1 and grown until the desired mycological biopolymer growth is achieved

### Example 4

1. A synthetically sourced and produced material, such as plastic and including but not limited to polyurethane, that contains the appropriate size, form, texture, and/or nutrition for mycelial growth, is used in its existing state or is cleaned through sterilization. Examples include but are not limited to packaging industry plastics, and furniture industry polyurethane or urethane.

2. Fungal inoculum is added to the synthetic base material.

3. The mixture is placed into a growth form designed to incite growth of mycological biopolymer material.

4. The filled growth form is placed into an environmental chamber as described in Example 1 and grown until the desired mycological biopolymer growth is achieved.

### Example 5

1. Any of the above Examples 1-4, but where the substrate base material size is altered to be larger than the ¼ standard in order to elicit specific growth morphologies or textures, such as bulbing, density differentiation, or gradation through mat height. Substrate base material size can range from just over to ¼ up to 2 inches, as well as to indeterminate size as a unibody structure, such as a contiguous mat or sheet of material that supports growth. Bulbing may be attained through inducing density variations in the substrate; density differentiation of the material may be obtained through localized substrate substitution; gradation through mat height may be obtained through the substrate with nutrition addition adjustment through the base material.

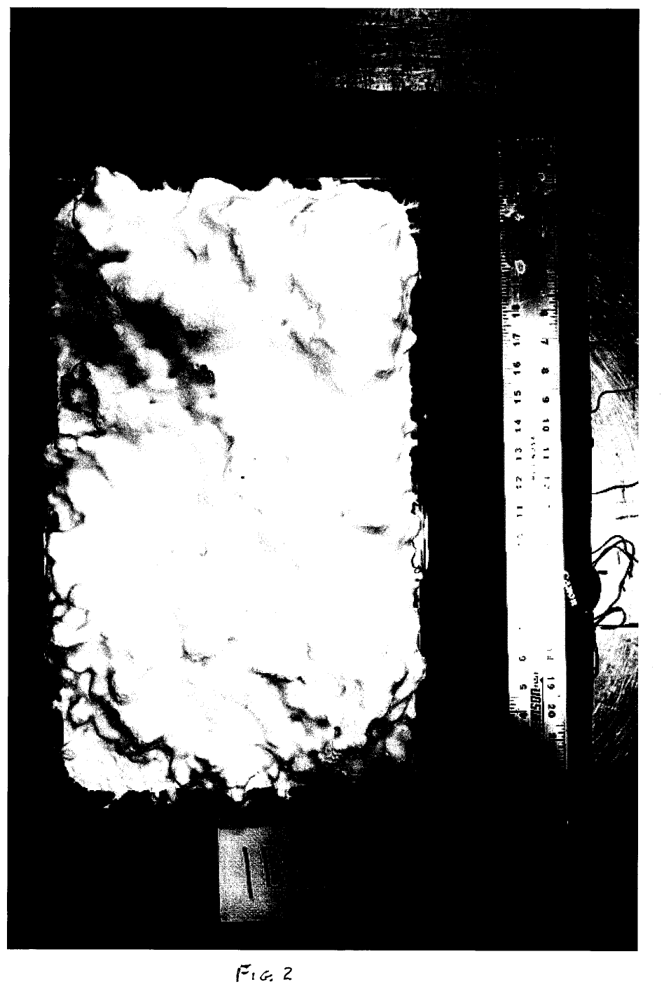

Referring to FIG. 2, the aerial mycelium growth produced according to Example 5 is characterized in having generally heterogeneously produced tissue texture. As such, the mycelial mat growth may function as a muscle tissue substitute for use in meat alternative production where variations in mycelium density capably replicate the variations naturally found in muscle tissue. As such, the aerial mycelial growth may function as an edible product

### Example 6

1. Any of the above Examples 1-4, but where inoculum size or type is altered from the standard millet particle size in order to elicit specific growth morphologies or textures, such as bulbing, density differentiation, or gradation through the mat height. An example includes maceration of Ganoderma species inoculated millet particles, which are generally at an initial size of 3mm in diameter, into smaller particulates, which results in a lower density lofted mycological biopolymer material than when standard millet size is utilized.

### Example 7

1. Any of the above Examples 1-2, but where the substrate base material is not subjected to cleaning through sterilization

### Example 8

1. Any of the above Examples 1-4, but where the substrate base material is altered during the growth cycle, either through self-decomposition, automated alteration, or physical manipulation, such as but not limited to, scratching, impaling, rearranging, or otherwise impacting change to the substrate homogeneity. Examples include self-decomposition through temporal deterioration; automated alteration by physical surface perturbation by machine; physical manipulation by physical surface perturbation by human hand where the substrate is scratched, impaled or rearranged using an appropriate tool such as scalpel, rod, or knife.

### Example 9

1. Any of the above Examples 1-8, but wherein nutrition addition is supplemented or altered to include supplementation during the growth period and not solely at the beginning of the growth cycle.

Examples include but are not limited to water addition to the substrate or growing tissue during the growth period to replenish that which was lost in order to maintain static levels best suited for tissue growth; nutrient addition as a solubilized spray or liquid to replenish that which was consumed in order to prevent lapse in tissue growth due to nutrient depletion.

### Example 10

1. Any of the above Examples 1-9, but where the mixture of substrate base, inoculum and nutrition are not placed into a growth form, but are instead applied to a growth surface or supportive base of any dimension.

Examples include any surface with sufficient strength to support the growth substrate and resultant mycelial mass, including but not limited to tables and conveyors. Additional examples include formless, cast materials, such as inverted forms that are ejected from their mold prior to initiating the growth cycle, whereas the angle of repose is maintained inherently by the supporting surface's structural integrity.

2. The growth surface is placed into an environmental chamber as described in Example 1 and grown until the desired mycological biopolymer growth is achieved.

### Example 11

1. Any of the above Examples 1-4, but with an even further reduced particle size of less than 0.125″ for an increase in uniformity of tissue grown from the material.

### Example 12

1. Example 1, but with wood processing performed specifically such that a whole tree is de-barked, chipped, reduced to wood flour, and then immediately dried and sealed in bags for storage in order to reduce the exposure of the substrate to any external environmental contamination. Wood flour is described as an industry standard particle size that falls between sieve mesh sizes of 80 to 100M.

Bags of substrate are then only opened immediately prior to use. This being different from the use of corn or other agricultural waste where the exterior of the plant—which has been exposed to prolonged periods of bioburden—is integrated into the final substrate.

### Example 13

1. Any of the above Examples 1-4, but where the substrate is specifically selected or formulated to have low quantities of heavy metals or other contaminants so as to grow a mycelium tissue with low quantities of such contaminants such as might be used in the food industry.

### Example 14

1. Any of the above Examples 1-4, but where the substrate is further doped with a specific compound, such as nitrites, supplements or a drug, which is then bio-accumulated by the mycelium tissue and becomes a functional component of the finished product, such as a food preservative or impregnated drug for wound dressing. An example includes, but is not limited to, supplementing with allicin, which is a health supplement/drug derived from garlic.

### Example 15

1. Example 3 where the material is agar media, specifically formulated through the addition of nutrition, including but not limited to, one or more drugs, supplements or minerals in order to support mycelial growth.

2. Example 3 where the material is agar media, and furthermore where the inoculation is across the top surface of the agar media rather than mixed throughout.

### Example 16

Any of the above Examples 1-4, wherein the nutritional addition is tailored chemically to elicit specific morphological changes in resulting mycological biopolymer material. Examples include but are not limited to increasing solubility of calcium addition, to decrease tissue density; supplementation with fatty acids, to increase growth rate; introduction of signaling molecules such as forskolin or 10-oxo-trans-8-decenoic acid, to increase hyphal branching and increase biopolymer density.

### Example 17

1. Example 7, wherein compounds inhibitory to unwanted contaminants are added to the non-sterile substrate matrix, to assist in desired fungal growth. Examples include but are not limited to the addition of tannins to retard ascomycetes, cinnamaldehyde to inhibit bacteria, or sorbate to inhibit yeast.

### Example 18

1. Any of the above Examples 1-4, wherein after extraction of the grown mycological biopolymer from the substrate surface upon which it was grown, the substrate is reused for a second growth flush, with or without additional nutritional amendment.

The invention thus provides alternative growth media to agricultural lignocellulosic waste for mycological biopolymer growth while also simplifying the growing of mycological biopolymers.

### Example 19

Referring to FIG. 3, the wet mass of aerial mycelial growth, referred to here as a panel, produced according to the Examples above will vary in dependence on the growth media, i.e. the substrate and fungus. For example, the wet mass of aerial mycelial growth produced according to Examples 1 and 2 varied as follows:

| Substrate-Organism | Wet panel mass (grams) |
|---|---|
| Corn stover-Ganoderma | 540 |
| Hemp-Ganoderma | 300 |
| Maple flour-Ganoderma | 530 |
| Corn cob-Ganoderma | 480 |
| Vermiculite-Ganoderma | 420 |
| Cellulose-Ganoderma | 510 |
| Corn stover-Pleurotus | 90 |
| Maple flour-Pleurotus | 75 |
| Corn cob-Pleurotus | 200 |
| Vermiculite-Pleurotus | 10 |

As compared to use of corn stover, the use of the alternative growth media as substrate and fungal mixture results in a wet panel mass of varying degrees depending on the growth media. As used herein, the term “wet panel mass” means the mass of the aerial mycelial growth after removal from the substrate and before drying.

Some nonlimiting embodiments of the disclosure are listed below.
1) A method of preparing a mycological biopolymer, comprising:

providing a mixture, said mixture comprising a substrate and a fungal inoculum, wherein said fungal inoculum comprises a fungus; and

incubating said mixture as a solid-state culture in a growth environment of moisture, temperature, carbon dioxide (CO2) and oxygen (O₂) sufficient to support fungal mycelial growth;

thereby providing a product comprising the mycological biopolymer.
2) The method of embodiment 1, wherein the incubation occurs over a period of time sufficient for said fungus to produce the product comprising the mycological biopolymer.
3) The method of embodiment 1 or 2, further comprising terminating the incubation prior to said fungus forming a visible fruiting body.
4) The method of embodiment 1, 2 or 3, wherein the mycological biopolymer product does not contain a visible fruiting body.
5) The method of embodiment 3 or 4, wherein the fruiting body is a stipe, pileus, gill or pore structure.
6) The method of any one of the preceding embodiments, wherein the product is an aerial mycological biopolymer.
7) The method of any one of the preceding embodiments, wherein the mycological biopolymer has a thickness of greater than about 0.125 inch.
8) The method of any one of the preceding embodiments, wherein the mycological biopolymer has a thickness of at least about 0.25 inch.
9) The method of any one of the preceding embodiments, wherein the mycological biopolymer has a thickness of at least about 0.5 inch, at least about 0.75 inch or at least about 1 inch.
10) The method of any one of the preceding embodiments, wherein the mycological biopolymer has a thickness of at most about 4, 6, 8 or 10 inches.
11) The method of any one of the preceding embodiments, wherein the mycological biopolymer is not appressed.
12) The method of any one of the preceding embodiments, wherein the substrate is a natural substrate.
13) The method of embodiment 12, wherein the natural substrate comprises a lignocellulosic material.
14) The method of embodiment 13, wherein the lignocellulosic material comprises a plant or wood material.
15) The method of embodiment 14, wherein the plant or wood material is purposefully harvested for use in the production of the mycological biopolymer.
16) The method of embodiment 14, wherein the lignocellulosic material is not an agricultural waste product.
17) The method of embodiment 13, 14 or 17, wherein the lignocellulosic material is not corn stover.
18) The method of embodiment 13, 14, 15 or 16, wherein the lignocellulosic material comprises hemp, maple, corn, kenaf, canola, soy straw, wheat straw, seed or seed husk material; or any combination thereof.
19) The method of embodiment 18, wherein the seed is selected from the group consisting of sunflower seed, walnut and poppy seed; and combinations thereof.
20) The method of embodiment 13, 14, 15 or 16, wherein the lignocellulosic material is a wood material comprising a hardwood or softwood material.
21) The method of embodiment 20, wherein the hardwood or softwood is of the genus Acer, Quercus, Populus, Abies or Pinus.
22) The method of any one of embodiments 13-17, wherein the lignocellulosic material comprises a wood flour or a plant flour.
23) The method of embodiment 22, wherein the wood flour is maple wood flour.
24) The method of embodiment 22, wherein the plant flour is soy flour.
25) The method of embodiment 12, wherein the natural substrate comprises a cellulosic material.
26) The method of embodiment 25, wherein the cellulosic material is a lignose-free material.
27) The method of embodiment 25, wherein the cellulosic material comprises plant fiber.
28) The method of embodiment 27, wherein the plant fiber is a fiber obtained from cotton (Gossypium sp.), hemp (Cannabis sp.), flax (Linum sp.) or jute (Corchorus sp.).
29) The method of embodiment 25, wherein the cellulosic material comprises pet bedding, paper, cardboard, card stock, cotton, linen or textile; or a combination thereof.
30) The method of embodiment 12, wherein the natural substrate comprises an inorganic material optionally, wherein the inorganic material is a mineral or mineral-based material.
31) The method of embodiment 30, wherein the inorganic material is a mineral or mineral-based material selected from the group consisting of vermiculite, perlite, soil, chalk, gypsum, clay, sand, rockwool and growstones; and combinations thereof; optionally, the clay is expanded clay or clay in the form of beads.
32) The method of embodiment 30, wherein the mineral or mineral-based material is a lignose-free material.
33) The method of any one of embodiments 1-11, wherein the substrate comprises a synthetic material.
34) The method of embodiment 33, wherein the synthetic material is a plastic.
35) The method of embodiment 33, wherein the synthetic material is a synthetic polymer.
36) The method of embodiment 35, wherein the synthetic polymer is a synthetic organic polymer.
37) The method of embodiment 36, wherein the synthetic organic polymer is selected from the group consisting of a polyethylene, a polypropylene, a polyvinyl chloride, a polystyrene, a polyacrylate, a nylon, a polytetrafluoroethylene (e.g.,Teflon™), a polyamide, a polyester, a polysulfide, a polycarbonate, a polythene or a polyurethane.
38) The method of embodiment 36, wherein the synthetic organic polymer contains one or more heteroatoms.
39) The method of embodiment 38, wherein the synthetic organic polymer containing one or more heteroatoms is selected from the group consisting of a polyamide, a polyester, a polyurethane, a polysulfide and a polycarbonate; optionally, the synthetic organic polymer is a polyurethane, which is optionally a thermoplastic polyurethane.
40) The method of any one of embodiments 33-39, wherein the synthetic material is obtained from a recycled material.
41) The method of any one of embodiments 1-11, wherein the substrate comprises an artificial material.
42) The method of embodiment 41, wherein the artificial material comprises alginate, rayon, agar or agar-agar; optionally the rayon is rayon fiber, such as Viscose.
43) The method of embodiment 42, wherein the alginate is sodium alginate.
44) The method of any one of the preceding embodiments, wherein the substrate is a sterile substrate.
45) The method of embodiment 44, wherein the method further comprises sterilizing the substrate prior to providing the mixture.
46) The method of embodiment 45, wherein the sterilization comprises heat sterilization, steam sterilization, or irradiation with electromagnetic radiation; optionally, the electromagnetic radiation comprises gamma rays, X-rays, UV or UV-visible radiation.
47) The method of any one of the preceding embodiments, wherein the substrate is provided as particles, said particles characterized as having a particle size.
48) The method of embodiment 47, wherein the particle size is at most about 0.25 inch in diameter.
49) The method of embodiment 45, wherein the particle size is less than 0.25 inch in diameter.
50) The method of embodiment 47, wherein the particle size is at most about 0.125 inch in diameter.
51) The method of embodiment 45, wherein the particle size is less than about 0.125 inch in diameter.
52) The method of embodiment 47, wherein the particle size is at most about 0.01 inches in diameter.
53) The method of embodiment 52, wherein the particle size is less than about 0.01 inch in diameter; optionally, at most about 0.007 inch in diameter.
54) The method of embodiment 47, wherein the particle size is at least about 0.25 inch, or greater than 0.25 inch in diameter.
55) The method of embodiment 54, wherein the particle size is at most about 2 inches in diameter.
56) The method of any one of embodiments 47 to 55, wherein the method further comprises sizing the substrate to said particle size prior to providing the mixture.
57) The method of any one of embodiments 1-46, wherein the substrate is a monolithic substrate.
58) The method of embodiment 57, wherein the monolithic substrate is a contiguous porous solid.
59) The method of embodiment 57 or 58, wherein the monolithic substrate is a log, a slab of wood, textile or a solidified porous gel medium; or a combination thereof.
60) The method of embodiment 57, 58 or 59, wherein the monolithic substrate is a continuous woven textile or a continuous non-woven textile.
61) The method of embodiment 60, wherein the continuous textile comprises rockwool, cotton (including nonwoven cotton), wood fiber or polyester fiber; optionally, the continuous textile is provided in the form of a mat.
62) The method of any one of embodiments 57-61, wherein the monolithic substrate comprises a combination of two or more monolithic substrates.
63) The method of embodiment 62, wherein the combination of the two or more monolithic substrates comprises the log slab penetrating into or through the solidified porous gel medium.
64) The method of any one of the preceding embodiments, wherein the substrate is a non-toxic substrate.
65) The method of any one of the preceding embodiments, wherein the fungus is of a type which produces edible mushrooms.
66) The method of embodiments 65, wherein the fungus is selected from the group consisting of Agaricus sp., Lentinula sp., Flammulina sp., Laetiporus sp., Pleurotus sp., Hericium sp., Morchella sp., Agrocybe sp. and Hypsizygous sp.
67) The method of embodiment 66, wherein the fungus is Pleurotus ostreatus.
68) The method of any one of embodiments 1-64, wherein the fungus is a white rot fungus.
69) The method of embodiment 68, wherein the fungus is selected from the group consisting of Phanerochaete sp., Ceriporiopsis sp., Ganoderma sp.,Omphalotus sp., Trametes sp., Polyporellus sp. and Schizophyllum sp.
70) The method of embodiment 69, wherein the fungus is Ganoderma tsugae or Ganoderma G. resinaceum.
71) The method of any one of the preceding embodiments, wherein the mixture further comprises one or more nutritional supplements.
72) The method of embodiment 71, wherein the nutritional supplement is a lignocellulosic material.
73) The method of embodiment 72, wherein the lignocellulosic material comprises seed, seed husks or both.
74) The method of embodiment 73, wherein the seed is sunflower seed, walnut, or poppy seed; or a combination thereof.
75) The method of any one of embodiments 1-70, wherein the mixture consists essentially of, or consists of, the substrate and the fungal inoculum.
76) The method of any one of the preceding embodiments, wherein providing the mixture comprises inoculating the substrate with the fungal inoculum.
77) The method of any one of embodiments 1-74, wherein providing the mixture comprises inoculating a blend containing the substrate and the nutritional supplement with the fungal inoculum to provide the mixture.
78) The method of any one of the preceding embodiments, wherein the fungal inoculum is a seed-supported fungal inoculum, a seed-saw dust mixture fungal inoculum, or another commercially available fungal inoculum (for example, a specialty proprietary spawn type provided by inoculum retailers).
79) The method of embodiment 78, wherein the seed-supported fungal inoculum has a density of about 0.1 gram per cubic inch to about 10 grams per cubic inch, or from about 1 gram per cubic inch to about 7 grams per cubic inch.
80) The method of any one of embodiments 3-79, wherein the termination of the incubation comprises exposing the incubation mixture comprising the solid-state culture to a terminal environment, wherein said terminal environment is different from the growth environment.
81) The method of embodiment 80, wherein said terminal environment has one or more conditions that differ from the corresponding growth environment conditions; optionally, the one or more environmental conditions is selected from the group consisting of moisture content, temperature, carbon dioxide content and oxygen content; and combinations thereof.
82) The method of embodiment 80 or 81, wherein exposing the incubation mixture comprising the solid-state culture to the terminal environment comprises physically moving said mixture comprising said solid-state culture from the growth environment to the terminal environment.
83) The method of embodiment 81, wherein exposing the incubation mixture comprising the solid-state culture to the terminal environment comprises modifying one or more conditions of the growth environment, thereby providing the terminal environment.
84) The method of embodiment 80 or 81, wherein terminating the incubation comprises restoring the growth environment to ambient environmental conditions.
85) The method of any one of embodiments 80-84, wherein the terminal environment is an ambient environment having ambient atmospheric environmental conditions.
86) The method of any one of the preceding embodiments, wherein the substrate is a solid or a gel.
87) The method of any one of embodiments 1-85, wherein the substrate is a liquid, provided that the culture is not a submerged culture.
88) The method of any one of the preceding embodiments, wherein the product consists essentially of, or consists of, the mycological biopolymer.
89) The method of any one of embodiments 1-87, wherein the product comprising the mycological biopolymer further comprises a portion of the substrate, nutritional supplement, or a combination thereof.
90) The method of any one of the preceding embodiments, further comprising removing the mycological biopolymer from at least a portion of the substrate.
91) The method of embodiment 90, wherein the method further comprises removing the mycological biopolymer from any remaining substrate.
92) The method of any one of the preceding embodiments, wherein the mixture comprising the fungal inoculum is incubated in a tool, wherein said tool is essentially as described in US2015/2015/0033620, the entire content of which is hereby incorporated by reference in its entirety.
93) The method of any one of the preceding embodiments, further comprising placing the mixture inside a tool, said tool comprising a container, wherein said mixture is placed inside said container.
94) The method of embodiment 93, wherein the tool further comprises a lid, said lid having an opening.
95) The method of any embodiment 93 or 94, wherein the container maintains a void volume after the placing of the mixture into the container.
96) The method of any one of embodiments 1-91, wherein the incubation of the mixture comprising the solid-state culture is performed on a planar surface.
97) The method of embodiment 94, wherein the surface is a tray, a sheet, a table or a conveyer belt.
98) The method of embodiment 96 or 97, wherein the method further comprises placing the mixture on the planar surface.
99) The method of embodiment 96, 97 or 98, wherein the planar surface is contained in a room, wherein said room provides the growth environment.
100) The method of any one of the preceding embodiments, wherein the carbon dioxide is present at a level ranging from about 3% (v/v) to about 7% (v/v).
101) The method of embodiment 100, wherein the carbon dioxide level ranges from about 5% (v/v) to about 7% (v/v).
102) The method of any one of the preceding embodiments, wherein the growth environment temperature ranges from about 55° F. to about 100° F.; optionally, from about 85° F. to about 95° F.
103) The method of any one of the preceding embodiments, wherein the period of time is up to about 3 weeks; optionally, up to about 2 weeks.
104) The method of any one of the preceding embodiments, wherein the growth environment of moisture, temperature, carbon dioxide (CO₂) and oxygen (O₂) are sufficient to support fungal growth, said fungal growth consisting essentially of fungal mycelial growth.
105) The method of any one of the preceding embodiments, wherein said fungal growth consists of mycelial growth.
106) The method of any one of the preceding embodiments, wherein the growth environment inhibits the formation of a fruiting body.
107) A product prepared by a method of any one of the preceding embodiments.
108) The product of embodiment 107, wherein the product is suitable for use as a foodstuff, provided that the fungus is the fungus of any one of embodiments 65-67.
109) Use of a product of embodiment 108 in the manufacture of a foodstuff.
110) The product of embodiment 107, wherein the product is suitable for use as a tissue scaffold.
111) Use of a product of embodiment 110 in the manufacture of a tissue scaffold.
112) Use of a product of embodiment 107 in the manufacture of a finished product.

### Other Embodiments

The various embodiments of systems, processes and apparatuses have been described herein by way of example only. It is contemplated that the features and limitations described in any one embodiment may be applied to any other embodiment herein, and flowcharts or examples relating to one embodiment may be combined with any other embodiment in a suitable manner, done in different orders, or done in parallel. It should be noted, the systems and/or methods described above may be applied to, or used in accordance with, other systems and/or methods. Various modifications and variations may be made to these example embodiments without departing from the spirit and scope of the embodiments, and the appended listing of embodiments should be given the broadest interpretation consistent with the description as a whole.

What is claimed is:

1. A method of growing a mycelial mat comprising the steps of
providing a growth medium of non-agricultural lignocellulosic waste media;
adding a nutrient for fungal growth and a fungal inoculum to said growth medium;
thereafter placing said growth medium in a closed incubation chamber;
maintaining said closed incubation chamber with a predetermined environment of humidity, temperature, carbon dioxide and oxygen sufficient to prevent the full differentiation of the fungus into a mushroom without producing a stipe, cap, or spores; and
incubating the growth media in each said container for a period of time sufficient for said fungus to digest said nutritive substrate and produce a mycelium mat consisting entirely of fungal mycelium on said growth medium.

2. A method as set forth in claim 1 wherein said growth media is a lignocellulosic material in particle size.

3. A method as set forth in claim 2 wherein said lignocellulosic material has a particle size of no more than ¼ inch.

4. A method as set forth in claim 2 wherein said lignocellulosic material has a particle size in a range of from ¼ inch to 2 inches.

5. A method as set forth in claim 1 wherein said growth media is a mineral based material.

6. A method as set forth in claim 5 wherein said mineral based material is at least one of vermiculite, perlite, soils and chalk.

7. A method as set forth in claim 1 wherein said growth media is a non-toxic non-cellulosic material.

8. A method as set forth in claim 7 wherein said non-toxic non-cellulosic material is one of at least one of hydroponic media, gels, polyacrylates, plastics and agars.

9. A method as set forth in claim 7 wherein said non-toxic non-cellulosic material is agar having an additive therein to support the growth of mycelium.

10. A method as set forth in claim 9 wherein said fungal inoculum is spread across said agar growth media.

11. A method as set forth in claim 1 wherein said growth media is a synthetically sourced and produced material.

12. A method as set forth in claim 11 wherein said material is a plastic.

13. A method as set forth in claim 12 wherein said material is polyurethane.

14. A method as set forth in claim 1 wherein said nutrient, fungus and growth medium are placed in at least one growth form prior to said step of placing said growth medium in a closed incubation chamber.

15. A method as set forth in claim 1 wherein said nutrient, fungus and growth medium are placed on at least one growth support surface prior to said step of placing said growth medium in a closed incubation chamber.

16. A method as set forth in claim 1 further comprising the step of doping said substrate with at least one of a nitrite, supplement and a drug.

17. A method as set forth in claim 16 wherein said supplement is a food preservative.

18. A method as set forth in claim 16 wherein said drug is allicin derived from garlic.

19. A method as set forth in claim 1 further comprising the step of adding a compound to said growth medium to inhibit contaminants.

20. A method as set forth in claim 19 wherein said compound is at least one of tannins to retard ascomycetes, cinnamaldehyde to inhibit bacteria, and sorbate to inhibit yeast.

21. A mycelium mat made in accordance with claim 1.
